(19) United States
(12) Patent Application Publication
(10) Pub. No.: US 2005/0112762 A1
(43) Pub. Date: May 26, 2005

(54) Method for culturing dendritic cells

(75) Inventors: Derek Nigel John Hart, Queensland (AU); Christopher Siaw Kang Ho, Queensland (AU); Jose Alejandro Lopez Ramirez, Queensland (AU)

Correspondence Address: Barry E Bretschneider;Morrison & Foerster, Suite 300, 1650 Tysons Boulevard, McLean, VA, 22102, US

(73) Assignee: The Corporation of the Trustees of the Sisters of Mercy in Queensland, South Brisbane (AU)

(21) Appl. No.: 10/496,871

(22) PCT Filed: Nov. 26, 2002
(86) PCT No.: PCT/AU02/01610

(51) Int. Cl. C12N 5/08

(52) U.S. Cl. 435/372

(57) ABSTRACT

The present invention relates generally to a cell culture process and to cells produced therefrom. More particularly, the present invention provides a method of supporting dendritic cell viability, proliferation and/or differentiation. The dendritic cells of the present invention are useful, inter alia, in the immunotherapy of cancer, infectious disease, autoimmunity and tumour therapy and as adjuvants, immune system modulating agents, immunotherapeutic agents and tolerising agents.

### FIELD OF THE INVENTION

The present invention relates generally to a cell culture process and to cells produced therefrom. More particularly, the present invention provides a method of supporting dendritic cell viability, proliferation and/or differentiation. The dendritic cells of the present invention are useful, inter alia, in the immunotherapy of cancer, infectious disease, autoimmunity and tumour therapy and as adjuvants, immune system modulating agents, immunotherapeutic agents and tolerising agents.

### BACKGROUND OF THE INVENTION

Bibliographic details of the publications referred to by author in this specification are collected at the end of the description.

The reference to any prior art in this specification is not, and should not be taken as, an acknowledgment or any form of suggestion that that prior alt forms part of the common general knowledge in Australia.

Dendritic cells (DCs) are a unique leukocyte population, which control the primary immune response (Hart D N J. Blood. 1997;90:3245-3287). They are extremely potent antigen presenting cells, distinguished by their exceptional ability to prime native T cells. They lack the expression of CD3, CD14, CD16, and CD19 molecules, but characteristically express high levels of major histocompatibility and co-stimulatory antigens. Two subsets of blood DCs have been described based on the differential expression of CD11c antigen (Kohrgruber N, Halanek N, Groger M, et al. J Immunol. 1999;163:3250-3259; Robinson S P, Patterson S, English A, et al. Eur J Immunol. 1999;29:2769-2778) and peanut agglutinin binding4. They appear to have distinctive characteristics and functions, including differential regulation by cytokines such as G-CSF and Flt3 ligand2. The classical CD11c⁺ “myeloid” DCs traffic into tissues and mucosal surfaces to act as immune sentinel cells and, after activation by pathogens or appropriate inflammatory stimuli, migrate via lymphatics to secondary lymphoid organs, where they initiate immune responses. The CD11c⁻ “lymphoid” DCs, also referred to as plasmacytoid or monocytoid lymphocyte-like DCs, express high levels of the CD123 antigen (interleukin-3 receptor α chain) on their surface. They are postulated to enter lymph nodes directly via the high endothelial venule to participate in immune responses (Celia M, Facchetti F, Lanzavecchia A, Colonna M. Nat Immunol. 2000;1:305-310).

Although there is general agreement that blood DCs are derived from hematopoietic stem cells, the concept that the different DC subsets may represent the progeny of different lineages remains controversial. The CD11c⁺ “myeloid” blood DCs, express the CD13 and CD33 myeloid differentiation antigens and include precursors for both epithelial and deep tissue (e.g. dermal) DCs. They depend on GM-CSF for survival in vitro (Kohrgruber N, Halanek N, Groger M, et al. J Immunol. 1999;163:3250-3259;Markowicz S, Engleman E G. J Clin Invest. 1990;85:955-961), but apparently not in vivo as DC populations are unchanged in GM-CSF deleted mice (Dranoff G, Crawford A D, Sadelain M, et al. Science. 1994;264:713-716). In contrast, the CD123^{hi} “lymphoid” DCs lack expression of CD13 and CD33, but express CD4 in greater amounts. Their survival in vitro is improved by the presence of IL-3 (Kohrgruber N, Halanek N, Groger M, et al. J Immunol. 1999;163:3250-3259;Dzionek A. Fuchs A, Schmidt P, et al. J. Immunol. 2000;165:6037-6046). Functionally, the CD11c⁺ DCs has the greater antigen uptake and immuno-stimulatory capacity (Robinson S P, Patterson S, English N. et al. Eur J Immunol. 1999;29:2769-2778), whereas the CD11c⁻ CD123^{hi} DCs has the ability to produce substantial amounts of interferon-α upon stimulation with pathogens (Siegal F P, Kadowaki N, Shodell M, et al. Science. 1999;284:1835-1837). The ability to produce DC like cells from monocytes cultured in GM-CSF plus IL-4 (and other mixtures) in vitro has introduced yet another potential DC precursor population into our considerations. There is some evidence that monocytes may generate into DCs in the tissue (Randolph G J, Inaba K, Robbiani D F, Steinman R M, Muller W A. Immunity. 1999;11:753-761) and we have postulated that this process provides a nascent boost to the antigen presenting cell populations in sites of significant infection or inflammation, rather than a primary route of differentiation for the myeloid DCs. In vitro studies on human DC differentiation plus studies using various relevant transcription factor deleted mice are beginning to investigate some of these complexities in DC differentiation.

Blood DCs are commonly purified for functional studies and similar preparations are also used in some clinical DC immunotherapy protocols (Fong L, Engleman E G. Annu Rev Immunol. 2000;18:245-273). It has been shown that activated blood DCs were better than freshly isolated DCs at processing, presenting and stimulating antigen specific T lymphocyte responses (Mannering S I, McKenzie J L, Hart D N. J Immunol Methods. 1998;219:69-83). However the ex vivo survival of isolated blood DCs, both CD11c⁺ and CD123^{hi}, is highly dependent on the support of cytokines, without which in vitro activation and culture, results in substantial loss of numbers.

Accordingly, there is an on-going need to develop effective and efficient methods of culturing dendritic cells in vitro such that their viability, proliferation and/or differentiation is maintained. In work leading up to the present invention, the inventors have determined that culturing DCs as part of a whole PBMC preparation, not only provides DC activation, but affords greater survival and thus recovery of functional blood DCs. Further, it has been determined that the subject PBMC cultures need not be supplemented with cytokines yet they nevertheless provide the DCs with the requisite physiological environment to support their survival, proliferation and/or differentiation in vitro.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention provides a method of supporting the viability, proliferation and/or differentiation of mammalian dendritic cells said method comprising culturing dendritic cells and/or precursors thereof in the presence of an effective number of mononuclear immune cells for a time and under conditions sufficient for said mononuclear immune cells to support said dendritic cells and/or precursors thereof.

Another aspect of the present invention more particularly provides a method of supporting the viability, proliferation and/or differentiation of mammalian dendritic cells said method comprising culturing dendritic cells and/or precursors thereof in the presence of an effective number of peripheral blood mononuclear cells for a time and under conditions sufficient for said peripheral blood mononuclear cells to support dendritic cells and/or precursors thereof.

Still another aspect of the present invention provides a method of supporting the viability, proliferation and/or differentiation of mammalian blood-derived dendritic cells said method comprising culturing said blood-derived dendritic cells aid/or precursors thereof in the presence of an effective number of peripheral blood mononuclear cells for a time and under conditions sufficient for said peripheral blood mononuclear cells to support said dendritic cells and/or precursors thereof.

Yet another aspect of the present invention provides a method of supporting the viability, proliferation and/or differentiation of mammalian CD14⁺/CD16⁺ blood-derived precursor cells said method comprising culturing blood-derived CD14⁺/CD16⁺ precursor cells in the presence of an effective number of peripheral blood mononuclear cells for a time and under conditions sufficient for said peripheral blood mononuclear cells to support said cell precursor cells.

In still another aspect there is provided the method of supporting the viability, proliferation and/or differentiation of mammalian blood-derived dendritic cells said method comprising culturing said dendritic cells and/or precursors thereof in the presence of an effective number of peripheral blood mononuclear cells for a time and under conditions sufficient for said peripheral blood mononuclear cells to support the viability of CD123^{hi}/CD11c⁻ dendritic cells and/or CD11c⁺ dendritic cells.

Still yet another aspect of the present invention provides a method of supporting the viability, proliferation and/or differentiation of mammalian blood-derived dendritic cells said method comprising culturing said dendritic cells and/or precursors in the presence of an effective number of peripheral blood mononuclear cells for a time and under conditions sufficient for said peripheral blood mononuclear cells to support the differentiation of CD14⁺/CD16⁺ precursor cells to Lin⁻ HLA-DR⁺ dendritic cells.

In yet still another aspect there is provided a method of supporting the viability, proliferation and/or differentiation of mammalian blood-derived dendritic cells said method comprising culturing said dendritic cells and/or precursors thereof in the presence of an effective number of peripheral blood mononuclear cells for a time and under conditions sufficient for said peripheral blood mononuclear cells to support the differentiation of said dendritic cells wherein expression of CD40, CD80, CD86, CMRF-44, CMRF-56 and/or CD83 is un-regulated.

In a further aspect there is provided a method of supporting the viability, proliferation and/or differentiation of mammalian blood-derived dendritic cells said method comprising culturing said dendritic cells and/or precursors thereof in the presence of an effective number of peripheral blood mononuclear cells for a time and under conditions sufficient for said peripheral blood mononuclear cells to support the proliferation of said dendritic cells and/or precursors thereof.

Another further aspect of the present invention contemplates a cell culture supernatant comprising tissue culture supernatant harvested from mononuclear immune cells cultured in accordance with the methods of the present invention wherein said cell culture supernatants supports the viability, proliferation and/or differentiation of mammalian dendritic cells and/or precursors thereof.

Still another further aspect of the present invention contemplates cell culture molecules comprising molecules harvested from mononuclear immune cells cultured in accordance with the method of the present invention wherein said cell culture molecules support the viability, proliferation and/or differentiation of mammalian dendritic cells and/or stromal cells.

Yet still another further aspect of the present invention contemplates a method of producing dendritic cells, said method comprising culturing dendritic cells and/or precursors thereof in the presence of an effective number of mononuclear immune cells for a time and under conditions sufficient to produce dendritic cells and/or precursors thereof.

A further aspect of the present invention contemplates the use of mononuclear imune cells in the manufacture of a cell culture system which is capable of supporting the viability, proliferation and/or differentiation of mammalian dendritic cells and/or precursors thereof.

In yet another aspect the present invention contemplates the use of dendritic cells generated in accordance with the method of the present invention in the manufacture of a medicament for the treatment of a mammal.

Another aspect of the present invention contemplates a method of therapeutically and/or prophylactically treating a subject said method comprising administering to said subject an effective number of dendritic cells wherein said dendritic cells are produced by the method of the present invention.

### BRIEF DESCRIPTION OF THE DRAWINGS

FIG. 1 is a graphical representation of the identification of blood DC in cultured PBMC. DCs were defined as cells that were negative for CD3, CD14, CD16, CD19, and CD34 (Lin⁻), and express HLA-DR (HLA-DR⁺); they represented a distinct cell population in cultured PBMC for up to 3 days (right-lower quadrants in A). The expression of CD40, CD80, CD86 co-stimulatory molecules (B), CMRF-44, CMRF-56 and CD83 activation markers (C), and CD11c⁺ and CD123⁺ subset markers (D) on gated Lin⁻ HLA-DR⁺ DCs was assessed using 3-color flow cytometry.

FIG. 2 is a graphical representation of the TruCOUNT™ analysis of absolute DC numbers in cultured PBMC. Dot plots demonstrate the forward—side scatter profile of cultured PBMC (A, left column). PBMC were gated in R1, TruCOUNT™ beads in R2 (A, left column), and Lin⁻ HLA-DR⁺ DCs in R3 (A, right column). Absolute DC numbers were calculated as the mean of triplicate determined by the number of Lin⁻ HLA-DR⁺ events per 10,000 TruCOUNT™ beads acquired for each time point (n=8, B, left). The filled and hatched portions of the bars represent the proportion of Lin⁻ HLA-DR^{lo} and Lin⁻ HLA-DR^{hi} cells in R3 respectively. PBMC were irradiated (3000 Gy) prior to culture (n=3, B, right). Error bars show SEM. (**, p<0.001).

FIG. 3 is a graphical representation of the TruCOUNT™ analysis of sorted DCs in culture with GM-CSF and IL-3. Dot plots demonstrate the forward—side scatter profile of sorted DC in culture supplemented with GM-CSF and IL-3 (A). Sorted Lin⁻ cells were gated in R1, and TruCOUNT™ beads in R2 (A). After gating for HLA-DR expression (R3, not shown), Lin⁻ HLA-DR⁺ DCs were analysed for their subset composition according to their expression of CD11c and CD123 (B). Absolute DC counts were calculated as the number of Lin⁻ HLA-DR⁺ cells per 10,000 TruCOUNT™ beads acquired (mean of triplicate, C, left). The absolute counts for DC subsets were calculated in similar fashion, based on the expression of CD11c (C, middle and right). This is representative of 4 separate experiments. For comparison, dot plots of a 4-color immunofluorescent FACS analysis of cultured PBMC (Lin.FITC, HLA-DR.PE-Cyanin5) demonstrated the persistence of both the CD11c⁺ and, more strikingly, the CD123⁺ DC subsets after 3 days of culture (D). Error bars show SEM. (*, p<0.05; **, p=0.09; **, p<0.001).

FIG. 4 is a graphical representation of the differential HLA-DR expression in DC within cultured PBMC. Using 4-color immunostaining the Lin⁻ HLA-DR^{high} (R4), and Lin⁻ HLA-DRdim (R5) in cultured PBMC (Day 1) were analysed with respect to the composition of the DC subsets (CD11c⁺ and CD123⁺). Isotype control for APC is shown. This pattern is representative of PBMC cultured for up to 3 days in 3 separate experiments.

FIG. 5 is a graphical representation of the kinetics of DC number change during PBMC culture. DC number (% PBMC) in cultured PBMC, with closer assessment in the first 24 hours period. Each time point was performed in triplicate. Error bars show SEM. This is representative of 4 separate experiments.

FIG. 6 is a graphical representation of the depletion experiments. PBMC are FACS-depleted of CD14, CD16, or CD19 cells, and cultured in parallel with whole PBMC samples. The percentage DC within the whole PBMC culture (▴, continuous line), CD14-depleted (▾), CD16-depleted (□), and CD19-depleted (⋄) was evaluated over a 24-hour period (3 experiments shown). Analyses at each time point were performed in triplicates. Error bars show SEM (A). Dot plots demonstrating the Lin⁻ HLA-DR⁺ DC profiles within the different depleted cultures at the 4-hour incubation time point from one experiment are shown (B), and they are representative of the 3 separate experiments performed.

FIG. 7 is a graphical representation of the antigen uptake by DC in cultured PBMC. DCs within the PBMC cultures were tested for their ability to take up F-Dx, LY, and F-TT fresh and after culture (A). The magnitude of antigen uptake was determined by FACS analysis and depicted as the mean ΔMFI (37° C.-4° C.)+SD (n=3). Open bars represent the HLA-DRhigh DC population (R4, FIG. 4), and the hatched bars represent the HLA-DRdim (R5, FIG. 4) population. Immature and LPS-matured Mo-DCs, fresh and cultured sorted DCs, and CMRF-44⁺ CD14⁻ CD19⁻ DCs, were tested for their F-Dx uptake capacity (B).

FIG. 8 is a graphical representation of the Allo-MLR of DCs sorted from fresh and cultured PBMC. DCs were sort purified from freshly isolated (●) and 3-day cultured (⋄) PBMC from the same donor, and tested for their ability to stimulate an allogeneic MLR. This is representative of 2 experiments.

### DETAILED DESCRIPTION OF THE INVENTION

The present invention is predicated, in part, on the determination that dendritic cell viability, proliferation and/or differentiation can be effectively and efficiently supported, without the addition of exogenous cytokines, where the subject dendritic cells are cultured together with peripheral blood mononuclear cells. The dendritic cells which are generated in accordance with the method of the present invention are useful in a range of therapeutic and/or prophylactic applications.

Accordingly, one aspect of the present invention provides a method of supporting the viability, proliferation and/or differentiation of mammalian dendritic cells said method comprising culturing dendritic cells and/or precursors thereof in the presence of an effective number of mononuclear immune cells for a time and under conditions sufficient for said mononuclear immune cells to support said dendritic cells and/or precursors thereof.

Reference to mononuclear immune cells which “support” the subject dendritic cells or precursors thereof should be understood to mean that the viability, proliferation and/or differentiation of a dendritic cell (or precursor cell thereof) is maintained and/or induced when the subject cell is cultured together with the mononuclear cells. It should be understood that the subject “support” is not necessarily provided indefinitely, to the extent that one maintains the dendritic cell-mononuclear cell co-culture, however it is at least transiently provided. This contrasts notably with the prior art methods which either could not at all provide such support or else provided such support only upon the application of complex exogenous growth factor regimes. Without limiting the present invention to any one theory of mode of action, it is thought that the subject mononuclear cells provide support by virtue of secreting growth factors and other soluble molecules and/or by cell-cell contact between the dendritic cell (or precursor thereof) and the mononuclear cell.

Reference to “mononuclear immune cells” should be understood as a reference to any mononuclear cells which either directly or indirectly function in the specific and/or non-specific immune response. Preferably, the subject mononuclear immune cells are a whole mononuclear cell population which has been isolated from a lymphoid organ or tissue such as blood, thymus, tonsil, lymph node, spleen or bone marrow. In a preferred embodiment, the subject mononuclear immune cells are peripheral blood mononuclear cells. In this regard, reference to “peripheral blood mononuclear cells” should be understood as a reference to any mononuclear cell which is either transiently or permanently located in the blood circulatory system. It should be understood that many of the mononuclear cells which can be found in the blood at any given point in time are actually recirculating cells. Accordingly, these cells are often only transiently present in the blood circulatory system and will also circulate in the lymphatic system and through the lymphoid organs (such as lymph nodes and spleen). Examples of peripheral blood mononuclear cells include, but are not limited to, recirculating T or B lymphocytes, monocytes, NK cells, dendritic cells, precursors thereof and committed and non-committed haematopoietic stem cells.

Accordingly, the present invention more particularly provides a method of supporting the viability, proliferation and/or differentiation of mammalian dendritic cells said method comprising culturing dendritic cells and/or precursors thereof in the presence of an effective number of peripheral blood mononuclear cells for a time and under conditions sufficient for said peripheral blood mononuclear cells to support dendritic cells and/or precursors thereof.

Reference to “peripheral blood mononuclear cells” should be understood to encompass both a whole peripheral blood mononuclear cell population or a sub-population thereof such as the lymphocyte component of the whole heterogeneous peripheral blood mononuclear cell population which is isolated from the donor. Further, it should be understood that the subject peripheral blood mononuclear cells may be only partially purified and may also comprise a contaminating portion of non-peripheral blood mononuclear cells, such as a granulocytes, which nevertheless do not adversely impact on the operation of the method of the present invention. It should be understood that an analogous definition applies to the mononuclear cell populations derived from other lymphoid tissues.

Reference to “dendritic cells” should be read as including reference to cells exhibiting dendritic cell morphology, phenotype or functional activity and to mutants or variants thereof. Reference to “dendritic cells” should be understood to include reference to cells at any differentiation state of development. The morphological features of dendritic cells may include, but are not limited to, long cytoplasmic processes, large cells with multiple fine dendrites (or other form of pseudopodia) or irregularly shaped membrane (although round cells are also observed). Phenotypic characteristics may include, but are not limited to, expression of one or more of the murine and human CD11c, CD123, MHC class II, CD1, CD4, CD8, Dec205, 33D1, CD80, CD86, CD83, CMRF-44, CMRF-56, DC-SIGN, DC-LAMP, Langerin or macrophage mannose receptor. Functional activity includes but is not limited to, a stimulatory capacity for naive allogeneic T cells, the capacity to internalise antigens and re-expressing peptides of said antigens in association with MHC Class II molecules. The expression of particular morphological, phenotypic and functional features will vary according to the differentiative state of the dendritic cell. For example, dendritic cells precursors are known to be effective as an antigen presenting cell. Expression of particular morphological, phenotypic and functional features may also vary between different populations of dendritic cells, such as dendritic cells arising from different cell lineages. For example, lymhoid-like dendritic cells vary from myeloid-like dendritic cells. “Variants” include, but are not limited to, cells exhibiting some but not all of the morphological or phenotypic features or functional activities of dendritic cells. “Mutants” include, but are not limited to, dendritic cells which are transgenic wherein said transgenic cells are engineered to express one or more genes such as genes encoding antigens, immune modulating agents, cytokines or receptors. Preferably, the subject dendritic cells are blood-derived dendritic cells.

The present invention therefore provides a method of supporting the viability, proliferation and/or differentiation of mammalian blood-derived dendritic cells said method comprising culturing said blood-derived dendritic cells and/or precursors thereof in the presence of an effective number of peripheral blood mononuclear cells for a time and under conditions sufficient for said peripheral blood mononuclear cells to support said dendritic cells and/or precursors thereof.

As detailed above, the subject dendritic cells may be at any differentiative stage of development. In this regard, reference to a dendritic cell “precursor” should be understood to include reference to any cell type which has the capacity, under appropriate conditions, to differentiate into a mature dendritic cell. Such a cell may be highly immature or it may be partially differentiated along either a dendritic cell or non-dendritic cell lineage, but which cell retains the capacity to complete its differentiation and or switch its differentiation, to a dendritic cell lineage. Highly immature cells, such as stem cells, or CFU cells, which retain the capacity to differentiate into a range of immune and non-immune cell types, should nevertheless be understood to satisfy the definition of “dendritic cell precursor” as utilised herein due to their capacity to differentiate into dendritic cells under appropriate conditions. In one example, the subject precursor dendritic cell is a CD14⁺/CD16⁺ blood-derived precursor cell.

Accordingly, in one embodiment the present invention provides a method of supporting the viability, proliferation and/or differentiation of mammalian CD14⁺/CD16⁺ blood-derived precursor cells said method comprising culturing blood-derived CD14⁺/CD 16⁺ precursor cells in the presence of an effective number of peripheral blood mononuclear cells for a time and under conditions sufficient for said peripheral blood mononuclear cells to support said cell precursor cells.

Methods for harvesting peripheral blood mononuclear cells would be well known to the person of skill in the art. For example, following harvesting of peripheral blood, peripheral blood mononuclear cells can be isolated by gradient separation, such as Ficoll-Hypaque centrifugation. Specifically, diluted anti-coagulated blood is layered over Ficoll-Hypaque and centrifuged. Red blood cells and polymorphonuclear leukocytes or granulocytes are more dense and centrifuge through the Ficoll-Hypaque, while mononuclear cells consisting of lymphocytes and monocytes band over it and can be recovered at the interface. Blood mononuclear cells may also be separated by elutriation.

It should be understood that the dendritic cells, precursor cells and peripheral blood mononuclear cells of the present invention may be in any suitable form. For example the cells may exist as a single cell suspension or they may comprise cell aggregates. The cells or aggregates may be derived from any suitable source. For example, the cells may be freshly isolated from an individual or from an existing cell line. The cells may be primary or secondary cells. A primary cell is one which has been isolated from an individual. A secondary cell is one which, following its isolation, has undergone some form of in vitro manipulation such as genetic manipulation. The cells may be derived directly from an individual or they may be derived from an in vitro source such as a tissue sample or organ which has been generated or synthesised in vitro. The subject cell or cell aggregate may also have been manipulated or stored subsequently to its isolation from a donor.

The process of the present invention may be “syngeneic”, “allogeneic” or “xenogeneic” with respect to the individuals within an animal species from which dendritic cells, precursors thereof and peripheral blood mononuclear cells are isolated. A “syngeneic” process means that the individual from which the dendritic cells are derived has the same MHC genotype as the origin of the peripheral blood mononuclear cells. For example, one individual's dendritic cells are co-cultured with the same individual's peripheral blood mononuclear cells. An “allogeneic” process is where the dendritic cells are from a MHC-incompatible individual from which the peripheral blood mononuclear cells are derived. For example, one individual's dendritic cells are co-cultured with another individual's peripheral blood mononuclear cells. A “xenogeneic” process is where the dendritic cells are from a different species to that from which the peripheral blood mononuclear cells are derived. For example, human derived dendritic cells are co-cultured with non-human primate derived peripheral blood mononuclear cells. Preferably, the method of the present invention is conducted as a syngeneic process. However, to the extent that either an allogeneic or xenogeneic process is utilised, it should be understood that it may be necessary to modify the co-culture such that any immunological responses which may occur due to the mixing of foreign immunocompetent cells is minimised. It would be within the skill of the person of skill in the art to design appropriate culturing parameters.

The method of the present invention contrasts notably with what has, to date, been the mandatory addition of cytokines to maintain even modest isolated blood dendritic cell survival (Kohrgruber N, Halanek N, Groger M, et al. J Immunol. 1999;163.3250-3259; Dzionek A, Fuchs A, Schmidt P, et al. J Immunol. 2000;165:6037-6046). In one embodiment of the present invention, the subject method supports the CD123^{hi}/CD11c⁻ subset and the CD11c⁺ subset of dendritic cells whereas control cultured isolated CD123^{hi} cells die rapidly. Without limiting the present invention in any way, it is thought that the natural production of cytokines and the cell-cell contact with the peripheral blood mononuclear cells provides the relevant survival signals to maintain dendritic cells.

Accordingly, in one embodiment there is provided the method of supporting the viability, proliferation and/or differentiation of mammalian blood-derived dendritic cells said method comprising culturing said dendritic cells and/or precursors thereof in the presence of an effective number of peripheral blood mononuclear cells for a time and under conditions sufficient for said peripheral blood mononuclear cells to support the viability of CD123^{hi}/CD11c⁻ dendritic cells and/or CD11c⁺ dendritic cells.

In another embodiment the method of the present invention supports the differentiation of CD14⁺ and/or CD16⁺ dendritic cells progenitors to Lin⁻ HLA-DR⁺ cells. Without limiting the present invention in any way, these Lin⁻ HLA-DR⁺ cells comprise a substantial proportion of CD11⁻ CD123^{hi} dendritic cells, thereby suggesting that these cells are derived from CD14⁺/CD16⁺ myeloid precursors.

Accordingly, in another embodiment of the present invention there is provided a method of supporting the viability, proliferation and/or differentiation of mammalian blood-derived dendritic cells said method comprising culturing said dendritic cells and/or precursors in the presence of an effective number of peripheral blood mononuclear cells for a time and under conditions sufficient for said peripheral blood mononuclear cells to support the differentiation of CD14⁺/CD16⁺ precursor cells to Lin⁻ HLA-DR⁺ dendritic cells.

Preferably, said Lin⁻/HLA-DR⁺ cells are CD11c⁻, CD123^{hi} dendritic cells or CD11⁺, CD123^{lo} dendritic cells.

Still without limiting the invention in any ways in another embodiment it is observed that the dendritic cells cultured in accordance with the method of the present invention up-regulate co-stimulatory (for example CD40, CD80, CD86) and/or activation (for example CMRF-44, CMRF-56 or CD83) markers.

Accordingly, in yet another embodiment there is provided a method of supporting the viability, proliferation and/or differentiation of mammalian blood-derived dendritic cells said method comprising culturing said dendritic cells aid/or precursors thereof in the presence of an effective number of peripheral blood mononuclear cells for a time and under conditions sufficient for said peripheral blood mononuclear cells to support the differentiation of said dendritic cells and/or precursors thereof wherein expression of CD40, CD80, CD86, CMRF-44, CMRF-56 and/or CD83 is up-regulated.

Preferably, said differentiated dendritic cells are CD11c⁻/CD123^{hi} dendritic cells and/or CD11c⁺ dendritic cells.

In still yet another preferred embodiment there is provided a method of supporting the viability, proliferation and/or differentiation of mammalian blood-derived dendritic cells said method comprising culturing said dendritic cells and/or precursors thereof in the presence of an effective number of peripheral blood mononuclear cells for a time and under conditions sufficient for said peripheral blood mononuclear cells to support the proliferation of said dendritic cells and/or precursors thereof.

Preferably, said proliferating dendritic cells are CD11c⁻/CD123^{hi} dendritic cells and/or CD11c⁺ dendritic cells.

The dendritic cells and the peripheral blood mononuclear cells of the present invention are co-cultured. By “co-culture” is meant the simultaneous culturing of two or more populations of cells, being, in accordance with the present invention, the dendritic cells (and/or precursors thereof) and the peripheral blood mononuclear cells. It should be understood that the subject cells will have been co-cultured provided that they were at least transiently co-cultured. That is, it may be desirable to culture one or both populations of cells, in isolation, either prior or subsequently to the co-culturing step. It should also be understood that during culture the subject cells may exist as non-adherent cells or they may become adherent.

The dendritic cells, precursors thereof and the peripheral blood mononuclear cells of the present invention are mammalian. The term “mammalian” is the adjectival form of “mammal” and includes humans, primates, livestock animals (eg. horses, cattle, sheep, pigs, donkeys), laboratory test animals (eg. mice, rats, rabbits, guinea pigs), companion animals (eg. dogs, cats) and captive wild animals (eg. kangaroos, deer, foxes). Preferably, the mammal is a human or laboratory test animal. Even more preferably the mammal is a human.

An “effective number” means a number necessary to at least partly obtain the desired outcome. The number varies depending upon the culture conditions and other relevant factors. It is expected that the number will fall in a relatively broad range which can be determined through routine trials.

Without limiting the present invention in any way, blood dendritic cells survive poorly in vitro when isolated from the peripheral blood mononuclear cell environment, even when cultured with cytokines such as GM-CSF and IL-3. However, when kept in contact with peripheral blood mononuclear cells, these dendritic cells survive for at least three days, in vitro, without the addition of exogenous cytokines. The relative percentage of Lin⁻ HLA-DR⁺ dendritic cells is maintained and, further, appear to separate into discreet HLA-DR^{hi} and HLA-DR^{lo} populations. An increment in the relative percentage of Lin⁻ HLA-DR⁺ dendritic cells is also observed following the first day of culture.

It should be understood that although the method of the present invention provides a culture system which does not require the addition of exogenous cytokines, the person of skill in the art would understand that the culture system disclosed herein may require the use of culture media supplements such as foetal calf serum, human serum, antibiotics, vitamins, amino acids (e.g. L-asparagine, L-arginine or glutamine), folic acid or sugars which are routinely used to provide a general optimisation of mammalian in vitro cell culture systems. However, it is also feasible that the method of the present invention maybe performed in serum free conditions.

In a most preferred embodiment, the method of the present invention is achieved via direct co-culture of dendritic cells (and or precursors thereof) with peripheral blood mononuclear cells. Such co-culture provides for both exposure of the dendritic cell to the growth factors and other soluble molecules produced by the peripheral blood mononuclear cells and cell-cell contact with the peripheral blood mononuclear cells. However, it should be understood that even in the absence of cell-cell contact, the supernatant produced by culturing the peripheral blood mononuclear cells is a valuable reagent in that it has been found to provide suitable support for dendritic cell viability, proliferation and/or differentiation.

Accordingly, another aspect of the present invention contemplates a cell culture supernatant comprising tissue culture supernatant harvested from mononuclear immune cells cultured in accordance with the methods of the present invention wherein said cell culture supernatants supports the viability, proliferation and/or differentiation of mammalian dendritic cells and/or precursors thereof.

Preferably said mononuclear immune cells are peripheral blood mononuclear cells and even more preferably said dendritic cells are blood-derived dendritic cells.

Reference herein to “cell culture supernatant” should be read as including reference to cell culture supernatant derived from cultures of mononuclear immune cells either together or separately from the subject dendritic cells or precursors thereof and includes functional derivatives and chemical equivalents of said supernatant. “Functional derivatives” include, but are not limited to fractions, homologues, analogues, mutants and variants having the functional activity of supporting the viability, proliferation and/or differentiation of dendritic cells and/or precursors thereof. This includes functional derivatives from natural or recombinant sources. “Chemical equivalents” can act as a functional analogue of said cell culture supernatant. Said chemical equivalents may be chemically synthesised or may be detected following, for example, natural product screening.

Another aspect of the present invention contemplates cell culture molecules comprising molecules harvested from mononuclear immune cells cultured in accordance with the method of the present invention wherein said cell culture molecules support the viability, proliferation and/or differentiation of mammalian dendritic cells and/or precursor cells thereof.

Preferably said mononuclear immune cells are peripheral blood mononuclear cells and even more preferably said dendritic cells are blood-derived dendritic cells.

Reference hereinafter to “cell culture molecules” should be read as including reference to molecules derived from cultures of mononuclear cells either together with or separately from the subject dendritic cells or precursors thereof and includes functional derivatives and chemical equivalents thereof. “Functional derivatives” include, but ale not limited to fractions, homologues, analogues, mutants and variants having the functional activity of supporting the viability, proliferation and/or differentiation of dendritic cells or precursors thereof. This includes functional derivatives from natural or recombinant sources. “Chemical equivalents” can act as a functional analogue of said cell culture molecules. Said chemical equivalents may be chemically synthesised or may be detected following, for example, natural product screening.

Said molecules are useful for inducing differentiation or proliferation or maintaining the viability of dendritic cells or precursors thereof.

Still another aspect of the present invention contemplates a method of producing dendritic cells, said method comprising culturing dendritic cells and/or precursors thereof in the presence of an effective number of mononuclear immune cells for a time and under conditions sufficient to produce dendritic cells and/or precursors thereof.

Preferably said dendritic cells are blood-derived dendritic cells and said mononuclear cells are peripheral blood mononuclear cells.

A further aspect of the present invention contemplates the use of mononuclear immune cells in the manufacture of a cell culture system which is capable of supporting the viability, proliferation and/or differentiation of mammalian dendritic cells and/or precursors thereof.

Preferably said dendritic cells are blood-derived dendritic cells and said mononuclear cells are peripheral blood mononuclear cells.

In yet another aspect the present invention contemplates the use of dendritic cells generated in accordance with the method of the present invention in the manufacture of a medicament for the treatment of a mammal.

Preferably said dendritic cells are blood-derived dendritic cells.

The methods and compositions of the present invention are useful for generating dendritic cells for use in a range of therapeutic and diagnostic procedures. For example, the dendritic cells may be used as cellular vectors for anti-tumour and infectious disease vaccines or as inducers of transplantation tolerance. These strategies are based on the highly developed antigen presenting capacity of dendritic cells. The dendritic cells of the present invention may also act as adjuvants for enhancing an immune response to, for example, tumour cells or other antigens such as those derived from prokaryotes, eukaryotes, or viruses, including human immuno deficiency viruses (e.g. HIV-I), influenza viruses and hepatitis viruses (e.g. Hepatitis A, B and C). Dendritic cells in a suitable state may also be loaded with such antigens to induce tolerance in transplantation or to treat autoimmune disease. The dendritic cells of the present invention may also be used as regulators of the immune response for example by down-regulating T cell activity or skewing the nature of a T cell response. Said down-regulation has applicability in the therapeutic or prophylactic treatment of disease conditions involving an unwanted immune response such as transplantation reactions (e.g. graft versus host disease) and autoimmune conditions (e.g. rheumatoid arthritis). Said skewing includes, for example, skewing a Th1 dominated immune response to a Th2 dominated immune response.

The dendritic cells produced by the method of the present invention may be fused with other cell types such as, for example, a tumour cell. Said fused dendritic cell is useful in a range of therapeutic and prophylactic procedures. For example, the processing and presentation of tumour antigens by a dendritic cell fused with a tumour cell has application as a therapeutic or prophylactic vaccine.

The cells produced by the methods of the present invention can also be used as immunogens for the production of monoclonal antibodies specific for new cell surface markers which define these cell types. Dendritic cells can be used for in vitro testing of the immunogenicity of vaccines. Dendritic cells can also be transplanted to various sites in an animal for effective migration or modulation of immune responses. Dendritic cells of the present invention can also be engineered to express foreign genes for effective gene therapy after transplantation and represent a long lived stem cell population which can colonise a host after transplantation, producing long tern reconstituting cells which can replicate and continue to express new genes.

Both the dendritic cells and the stromal cells of the present invention can be used to isolate genes and proteins expressed specifically by these cell types. To date, this has been an activity greatly limited by the availability of dendritic cells. The method of the present invention now facilitates the routine generation of dendritic cells thereby facilitating the performance of monoclonal antibody or gene assays. Further, the method of the present invention provides a useful in vitro source of dendritic cells for a large range of applications including, but not limited to:
(i) dendritic cell studies relating to stimulation, tolerance, induction and Th1/Th2 manipulation.
(ii) as a source of known and novel growth factors/cytokines.
(iii) the establishment of a system for the discovery of new dendritic cell associated molecules.
(iv) tracking dendritic cell differentiation.

Another aspect of the present invention contemplates a method of therapeutically and/or prophylactically treating a subject said method comprising administering to said subject an effective number of dendritic cells wherein said dendritic cells are produced by the method of the present invention.

Reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition. Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

The present invention is further defined by the following non-limiting Examples.

### EXAMPLE 1

### Spontaneous Generation and Survival of Blood Dendritic Cells in Mononuclear Cell Culture Without Exogenous Cytokines

Monoclonal Antibodies and Reagents

The following monoclonal antibodies (mAbs) were used: PE-conjugated CD3, CD14, CD16, CD19, CD34, CD7, CD80, and CD11c were obtained from Becton Dickinson (BD) (San Jose, Calif.); CD86, CD123, IgG1, and IgG2b isotype control were purchased from PharMingen (San Diego, Calif.); CD20, CD56, CD40, and CD83 from Coulter-Immunotech (Marseille, France); and CD64 from Serotec (Oxford, UK). FITC-conjugated CD11c was purchased from Serotec; CD2 from Coulter-Immunotech; and cutaneous leukocyte antigen (CLA) from PharMingen. PE.Cy5 (PE.Cy5)-conjugated HLA-DR from Coulter-Immunotech, and IgG1 isotype control from PharMingen; APC-conjugated CD11c and IgG1 isotype control tom BD; unconjugated mAbs—CD3 (OKT3), CD11b (OKM1) were obtained from the American Type Tissue Collection (ATTC, Rockville, Md.); CD16 (HuNK2), CD19 (FMC63) were gifts from Prof H Zola (Adelaide, Australia); CD34 from BD, CD14 (CMRF-31), CMR-44 (IgM), CMRF-56 (IgG1), and IgG1 (401.21), IgG2a (CMRF-84), and IgM (CM-50) isotype controls were produced at the Mater Medical Research Institute (Brisbane, Australia).

FITC-conjugated sheep anti-mouse immunoglobulin (FITC-SAM) was purchased from Amrad Biotech (Victoria, Australia); PE.Cy5-conjugated streptavidin from Dalco (Carpinteria, Calif.); mouse serum, 7-amino-actinomycin-D (7-AAD), propidium iodide (PI), and lipopolysaccharide (LPS) were purchased from Sigma-Aldrich (St Louis, Mo.). FITC-Dextran (F-Dx, M_{f}=42,000) and lucifer yellow (LY) CH dipotassium salt from Sigma-Aldrich were obtained as lyophilized powder and freshly reconstituted in medium prior to use. Tetanus toxoid (TT) was obtained from Commonwealth Serum Laboratories (Melbourne, Australia). TT was labeled by dialysis with FITC (Sigma-Aldrich) in 0.5 M bicarbonate buffer (pH 9.5) for 48 hours. The FITC to TT ratio determined by spectrophotometer (280 nm) was 9.6:1.

Recombinant human (rh) GM-CSF was obtained from Sandoz-Pharnia (Sydney, Australia), rh interleukin (IL)-3 from Gibco—Life Technologies (Melbourne, Australia), rhIL-4 (Sigma-Aldrich), and rhTNF-α from Hoffman-La Roche (Basel, Switzerland).

Complete media included RPMI 1640 with 10% FCS supplemented with penicillin (100 U/ml), streptomycin (100 μg/ml), L-glutamine (2 mM), and non-essential amino acids, all purchased from Gibco—Life Technologies. For antigen uptake experiments, the media also contained 25 mM HEPES (Gibco—Life Technologies).

Cell Preparation and Culture

PBMC were prepared from either whole blood from healthy volunteers or buffy coats (Australian Red Cross Blood Service, Brisbane) by standard Ficoll-Paque (Pharmacia, Uppsala, Sweden) density gradient centrifugation.

Blood DCs were obtained from PBMC by a two-step purification method as described previously with minor modifications (Fearnley D B, McLellan A D, Mannering S I, Hock B D, Hart D N J. Blood. 1997;89:3708-3716). Briefly, T cells, B cells, monocytes, and NK cells were depleted using immunomagnetic cell separation (Biomag™ beads, Polysciences, Warrington, Pa., and Variomacs™, Miltenyi Biotech, Gladbach, Germany) with antibodies specific for other hematopoietic lineages: CD3, CD19, CD14, CD11b, and CD16. To remove any remaining “lineage” positive cells after the depletion procedure, this cell preparation was labeled with FITC-SAM, blocked with mouse serum, stained with PE-conjugated antibodies specific for CD7, CD20, CD34, CD56, and CD64, and then purified using flow cytometric sorting (FACS Vantage, BD) for cells that were negative for FITC and PE signals.

Monocyte-derived DCs (Mo-DCs) were generated using the adherence method as described previously (Sallusto P. Cella M, Danieli C, Lanzaveechia A. J Exp Med. 1995;182:389-400). Briefly, PBMC were plated in 80-cm² Nunclon™ tissue culture flasks (Nunc, Roskilde, Denmark) and incubated for 2 hours. Nonadherent cells were removed and the remaining cells were cultured in complete media with GM-CSF (200 U/ml) and IL-4 (50 U/ml) for 5 days (Vuckovic S, Fearnley D B, Mannering S I, et al. Exp Hematol. 1998;26:1255-1264) to produce immature Mo-DCs, which were CD1a⁺ CD14⁻ CD83-(data not shown). These were cultured in the presence of LPS (μg/ml) for a further 2 days to generate CD1a⁺ CD14⁻ CD83⁺ mature Mo-DCs (Vuckovic S, Fearnley D B, Mannering S I, et al. Exp Hematol. 1998;26:1255-1264). T cells were obtained from PBMC using the sheep erythrocyte resetting method (Fearnley D B, McLellan A D, Mannering S I, Hock B D, Hart D N J. Blood. 1997,89:3708-3716), and were >93% CD3⁺.

Flow Cytometric Analysis

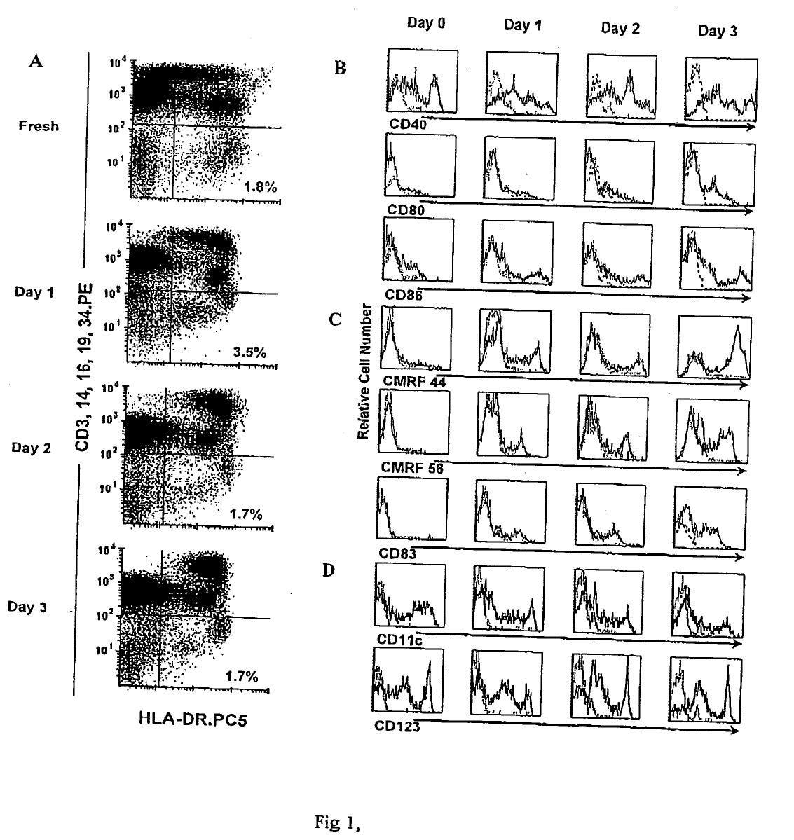

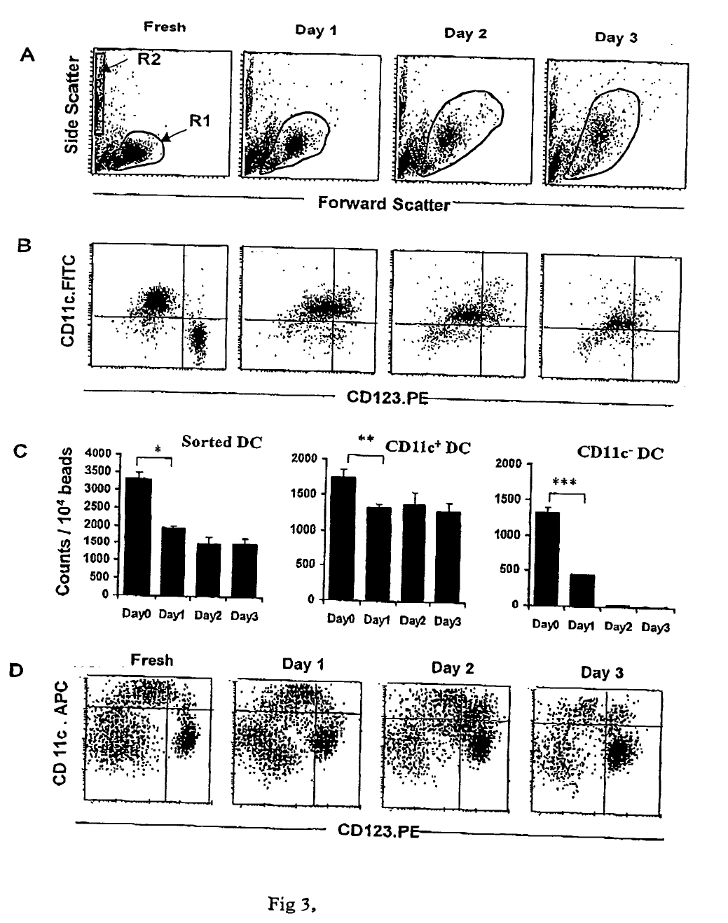

To analyze DCs in fresh and cultured PBMC, cells were stained with PE-conjugated lineage specific mAbs (CD3, CD14, CD16, CD19) and CD34, and PE-Cy5-conjugated HLA-DR mAb (FIGS. 1A, 2A, 6B). CD34 was added to the lineage mixture to exclude circulating hematopoietic stem cell population (MacDonald K P A, Munster D, Clark G, Vuckovic S, Hart D N J: Peripheral blood dendritic cell subset analysis, in Mason D (ed): Leucocyte Typing VII, vol. 7. Oxford, Oxford University Press, 2001). The expression of CMRF-44 and -56 was analyzed in the FITC channel, For other three-color immunofluorescence staining of PBMC, the combination of FITC fluorochrome for lineage markers, PE-Cy5 for HLA-DR, and PE for DC co-stimulatory (CD40, CD80, CD86), CD83 activation, and CD123 and CD11c subset molecules, was used (FIGS. 1B&D). For each analysis, 3×10⁵-10⁶ events were collected within the mononuclear gate. To further define the DC subsets in PBMC, four-color flow cytometric analysis using the APC channel for CD11c (FIG. 3D&4) was employed. Sorted Lin⁻ cells were gated for HLA-DR staining (PE-Cy5), and then analyzed for their expression of CD11 c (FITC) and CD123 (PE). Analysis was performed on a FACS Calibur flow cytometer (Becton Dickinson) using CellQuest software. Data was analyzed using either CellQuest 3.1 or FCS Express software.

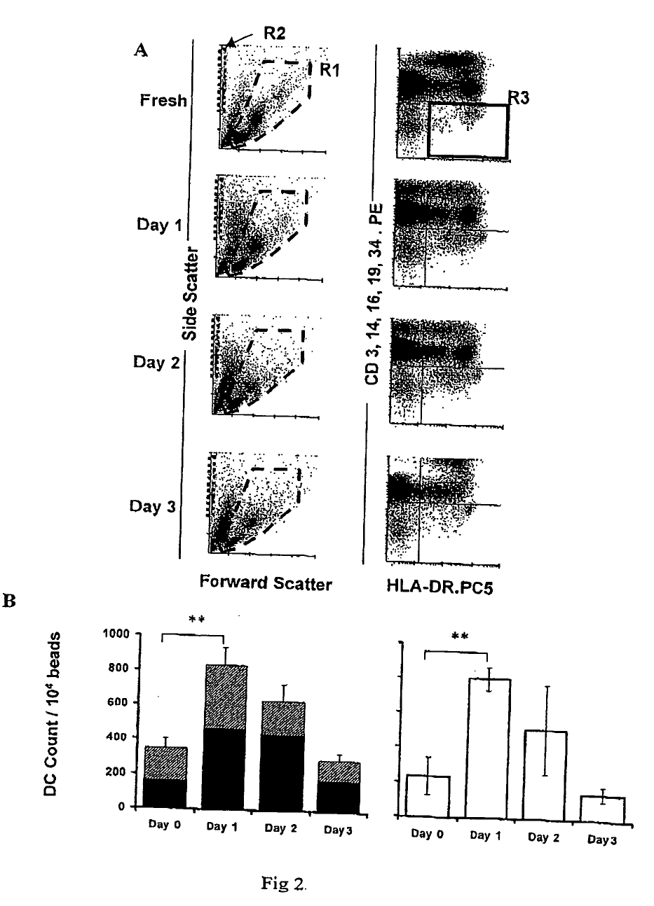

Viable cells were gated based on forward and side scatter characteristics (R1, FIG. 2A). Within the R1 gate, >99.5% of the cellular events in PBMC and sorted DCs, fresh and cultured (to 3 days) were negative for 7-AAD or PI when labeled (data not shown).

TruCOUNT™ Analysis of Absolute Cell Counts

TruCOUNT™ tubes (Becton Dickinson) were used to determine the absolute counts of DCs in PBMC cultures. Each tube contained a lyophilized pellet that dissolves releasing a known number of fluorescent beads. The tubes were used according to manufacturer's recommendations with minor modifications (Nicholson J K, Stein D, Mui T, et al. Clin Diagn Lab Immunol. 1997;4:309-313). PBMC were seeded and cultured in 96-well flat-bottom plates at 10⁷ cells/ml (200 μl/well), and harvested for staining at the various time points. The antibody mixture (CD3, CD14, CD16, CD19, CD34)-PE, and HLA-DR.PE-Cy5 was prepared for the experiment at a 1:30 dilution first, to ensure a consistent concentration of antibodies for each analysis. Then, 30 μl of the antibody mix was added to the TruCOUNT™ tube, followed by 20 μl of cells from the wells. The tube was vortexed gently to mix and incubated in the dark at room temperature for 15 minutes. Finally, 350 μl of PBS was added making a total volume of 400 μl prior to FACS analysis. A minimum of 500 Lin⁻ HLA-DR⁺ DC events (R3, FIG. 2A), and/or 20,000-35,000 beads (R2, FIG. 2A) were acquired for each analysis. Each sample was performed in triplicate. The absolute number of DCs in each sample was calculated as the average of the triplicate tubes, each being determined by comparing the cellular events (R3) to bead events (R2), and expressed as DC counts/10⁴ beads.

Depletion Assays

Single cell lineages or populations in PBMC were labeled using the PE-conjugated mAb: CD14, CD16, CD19, or CD3 respectively. Each cell population was depleted by FACS sorting and cultured in parallel with unseparated PBMC, as the positive control for each separate experiment. The cultured cells were labeled and analyzed by FACS to assess the percentage of DC present at predetermined time points of the culture period.

Antigen Uptake Assays

PBMC were seeded in 6-well plates at 10⁷ cells/ml for culture, harvested each day, and resuspended in pulsing medium for incubation with the antigens. F-Dx (1 mg/ml), LY (1 mg/ml), or F-TT (0.5 mg/ml) was added and incubated with the cells either at 4° C. (control) or 37° C. for 60 minutes. Cells were washed 4 times in cold PBS, then stained with the antibody mixture (CD3, CD14, CD16, CD19, CD34)-PE and HLA-DR.PE-Cy5, and analyzed immediately by FACS. The level of antigen uptake by DCs was assessed on the FITC channel after gating for the Lin⁻ HLA-DR⁺ cells, and expressed as the difference in mean fluorescence intensity (ΔMFI) between the test (37° C.) and control (4° C.) tubes for each sample.

Allogeneic Mixed Leukocyte Reaction (MLR)

Sorted DCs (10-20,000 cells) were incubated with allogeneic T-lymphocytes (10⁵ cells) for days in 96-well U-bottom plates. Sixteen hours prior to harvesting the cells, 18.5 kBq of ³H-thymidine was added to each well. ³H-thymidine uptake was counted in a liquie β-scintillation counter (Wallac, MicroBeta Trilux Scintillation Counter, Turku, Finland).

### EXAMPLE 2

### Blood DCs Survive in Cultured PBMC Without Exogenous Cytokines

Blood DCs were defined within PBMC by two-color flow cytometric analysis as HLA-DR⁺ cells that were lineage (CD3, CD14, CD16, CD19, and CD34) negative. Sorted blood DCs survived poorly in vitro when isolated from the PBMC environment, even when cultured with the cytokines GM-CSF and IL-3 as has been experienced before (Kohrgruber N, Halanek N, Groger M, et al. J Immunol. 1999;163:3250-3259;Dzionek A, Fuchs A, Schmidt P, et al. J Immunol. 2000;165:6037-6046). However, it was found that when kept in contact with the other PBMC, the DCs survived for at least 3 days, in vitro, without the addition of exogenous cytokines (FIG. 1A). The relative percentage of Lin⁻ HLA-DR⁺ DCs in PBMC was the same at the end of a 3-day culture as at its initiation (n=10). The Lin⁻ HLA-DR⁺ DCs in cultured PBMC appeared to separate into discrete HLA-DR^{hi} and HLA-DR^{lo} populations compared to the more homogeneous profile obtained when examined immediately ex vivo (FIG. 1A). An apparent increment in the relative percentage of Lin⁻ HLA-DR⁺ DCs was also noted on day 1 (see next section). When parallel experiments (n=3) were performed using x-vivo 10 (a serum free medium), the same phenomenon was observed. There was no statistically significant difference between the two culture systems (days 1-3, p>0.7).

These cultured PBMC DCs were analysed for their expression of the co-stimulatory molecules (CD40, CD80 and CD86) and the activation markets (CMRF 44, CMRF 56 and CD83). DCs within the PBMC cultures spontaneously and progressively upregulated these molecules in culture (FIGS. 1B and 1C). Both DC subsets, defined by the CD11c and CD123 molecules, were maintained in PBMC throughout the culture period (FIG. 1D) although the CD11c⁺ DC population upregulated its expression of the CD123 antigen (FIGS. 3B and 3D). The Lin⁻ HLA-DR⁺ DCs analyzed in fresh PBMC and after overnight culture, also expressed CLA and CD2 (data not shown). When LPS (10 ng/ml) or TNF-α (10 ng/ml) was added to the culture on Day 0, the expression of co-stimulatory molecules (CD40, CD86) and activation markers (CMRF-44, CD83) on DCs was higher (MFI) than would otherwise be seen after overnight culture, and equaled that cultured for 2 days (data not shown).

### EXAMPLE 3

### TruCOUNT™ Analysis Quantifies Rise of Absolute DC Counts in Cultured PBMC

The definite but variable increase in the percentage of DCs in PBMC noted after overnight (16-24 hour) culture (n=10) was investigated further. To assess whether the increase in number reflected an increase in absolute DCs or differential survival with respect to the other PBMC populations in culture, TruCOUNT™ beads (FIG. 2A) were used to obtain absolute DC counts in 8 further experiments. The TruCOUNT™ analysis confirmed a significant rise in absolute counts of Lin⁻ HLA-DR⁺ events after the overnight culture period (p<0.001, n=8, FIG. 2B left), and showed a close correlation between the changes in percentage DC number and absolute cell counts (not shown). The HLA-DR^{lo} DC population increased by 235%±77% (SEM) compared with 150%±45% (SEM) in the HLA-DR^{hi} population (FIG. 2B left).

To exclude the possibility that DC or DC precursor proliferation during the culture period was responsible for the increase, fresh PBMC were irradiated (3000 Gy), then cultured and analyzed, in parallel with their non-irradiated controls, again using TruCOUNT™ beads. Irradiation of the starting PBMC preparation did not affect the rise of absolute DC counts (n=3): similar increases in absolute Lin⁻ HLA-D⁺ DCs occurred in both instances (FIG. 2B), indicating that proliferation of DC precursors was not contributory.

### EXAMPLE 4

### Isolated DCS Survive Poorly in Culture as Determined by TruCOUNT™ Analysis

Using the TruCOUNT™ assay, confirmed previous data (Kohrgruber N, Halanek N, Groger M, et al. J. Immunol. 1999;163:3250-3259;Dzionek A, Fuchs A, Schmidt P, et al. J. Immunol. 2000;165:6037-6046) were confirmed indicating that isolated DCs survive poorly, even when cultured with GM-CSF and IL-3. Sorted Lin⁻ cells increased in size with culture as indicated by changes in the Side Scatter profile (FIG. 3A). The (HLA-DR⁺) CD11c⁺ DC subset can be easily distinguished from the (HLA-DR⁺) CD11c⁻ CD123^{hi} population immediately ex vivo, but after overnight culture, some of the CD11c⁺ cells, which were CD123^{lo}, upregulated the intensity of CD123 expression (FIG. 3B), whilst CD11c⁻ CD123^{hi} cells died rapidly. In freshly isolated DCs, <1% were double positive i.e. CD11⁺ CD123^{hi}, but this rises to 33% after overnight incubation. The isolated CD11c⁺ DC subset survived better (74-78% starting cells on Days 1 to 3), whereas the isolated CD11c⁻ CD123^{hi} DC population proportion dropped sharply to 34% starting cells after overnight culture, and to 2% and 1% of the original cells on Days 2 and 3, respectively (FIGS. 3B & 3C).

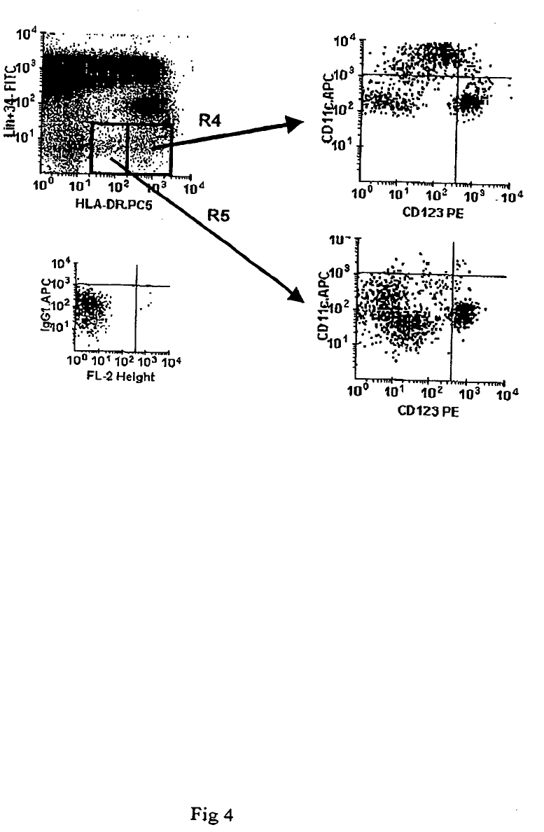

Because the CD11c⁺ DC subset upregulated CD123 antigen expression (FIG. 3B), 4-colour flow cytometry was used to define the two DC subsets in cultured PBMC accurately. The CD11c⁻ CD123^{hi} DC subset persisted and remained as a discrete population throughout the 3-day culture period (FIG. 3D), unlike the sorted CD11c⁻ CD123^{hi} DCs (FIGS. 3B & C). It was also noted that a CD11c⁺ CD123^{hi} population emerged during culture (FIG. 3D), as predicted by the experience with cultured sorted CD11c⁺ DCs (FIG. 3B). As mentioned earlier, the DCs in cultured PBMC expressed different levels of cell surface HLA-DR (FIGS. 1A & 4). The HLA-DR^{hi} population contained both the CD11c⁺ and CD11c⁻ CD123^{hi} DC subsets, whereas the HLA-DR^{lo} population contained more CD123^{hi} DCs (FIG. 4). This observation was the same for sorted Lin⁻ DCs (data not shown).

### EXAMPLE 5

### DC Numbers Increase Rapidly in Cultured PBMC

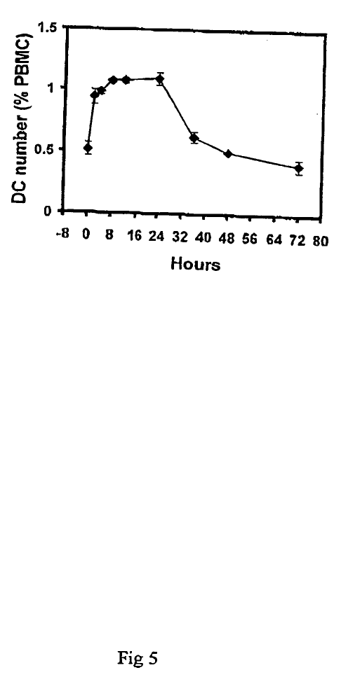

Since the rise in Lin⁻ HLA-DR⁺ DCs occurred mainly within the first 24 hours, this phenomenon was evaluated more closely. The increase was rapid and occurred within the first 4 hours of incubation, with the peak and plateau attained after 8-12 hours of incubation (FIG. 5). The DC number returned to baseline level after 48 hours of culture (FIG. 5). Taken together with the cell irradiation experiments above, this suggested that the increase in cell numbers was due to the contribution of either a population of Lin⁺ cells (down regulating their markers) or a population of Lin⁻ HLA-DR⁻ cells, upregulating HLA-DR expression to enter the Lin⁻ HLA-DR⁺ DC pool upon culture in vitro.

### EXAMPLE 6

### PBMC CD14⁺ and CD16⁺ Cells Contribute to the LIN⁻ HLA-DR⁺ DC Pool During Culture In Vitro

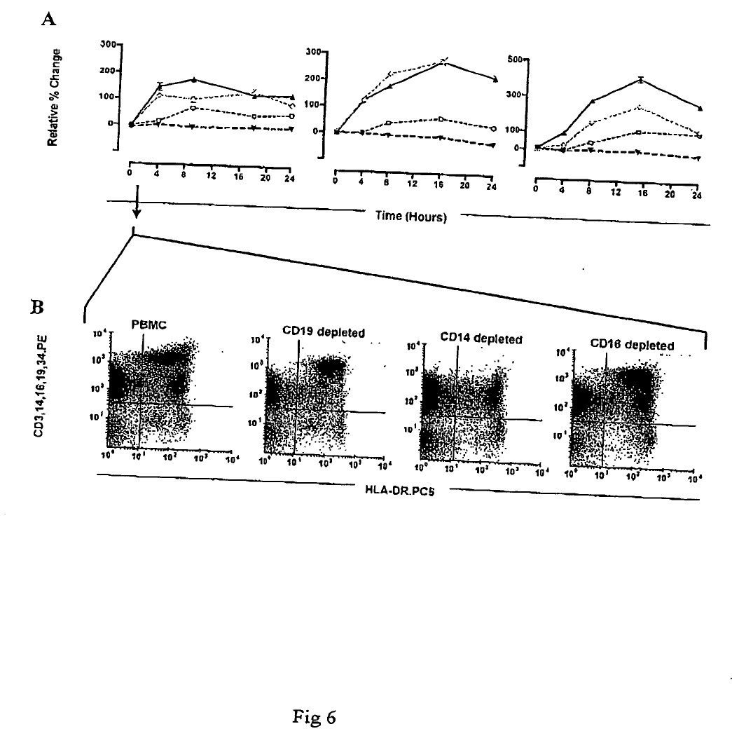

To address the origin of the increase in Lin⁻ HLA-DR⁺ DCs, a series of depletion experiments were performed. Single cell populations were removed from PBMC using appropriate CD markers (CD19, 14, 16 and 3), and each depleted PBMC population was then cultured in parallel with the control starting whole PBMC preparation. The Lin⁻ HLA-DR⁺ gate was used to follow changes in cell numbers and cell dot plot profiles. In contrast to the characteristic rise in DC numbers in whole PBMC cultures (FIG. 6), the Lin⁻ HLA-DR⁺ DC number remained remarkably constant in the PBMC cultures that were depleted of CD14⁺ monocytes (FIG. 6). In the CD16⁺ cell-depleted cultures, the Lin⁻ HLA-DR⁺ DC number only rose after the 4-hour time point, and this rise was considerably attenuated (FIG. 6). When CD19⁺ B cells were depleted, the effect was minimal (FIG. 6). Similarly, the removal of CD3⁺ T cells from the culture system did not affect the rise in DC number (n=2, not shown). This clearly demonstrated that the rapid and spontaneous in vitro emergence of “new” Lin⁻ HLA-DR⁺ cells required the presence of CD14⁺ and/or CD16⁺ PBMC in the culture.

The “new” Lin⁻ HLA-DR⁺ population was mainly in the HLA-DR^{lo} region of the DC gate. After a period of culture, the Lin⁻ HLA-DR⁺ cells showed clear separation into 2 clusters based on their HLA-DR expression (FIGS. 1A, 4 & 6). This phenomenon was absent when PBMC were either depleted of CD14⁺ or CD16⁺ cells, (FIG. 6B, 4 hour culture), suggesting that the “new” Lin⁻ HLA-DR⁺ cells were predominantly represented in the HLA-DR^{lo} region of the DC gate.

### EXAMPLE 7

### Activated DCS Increase Dextran but Decrease Tetanus Toxoid Uptake Capacity

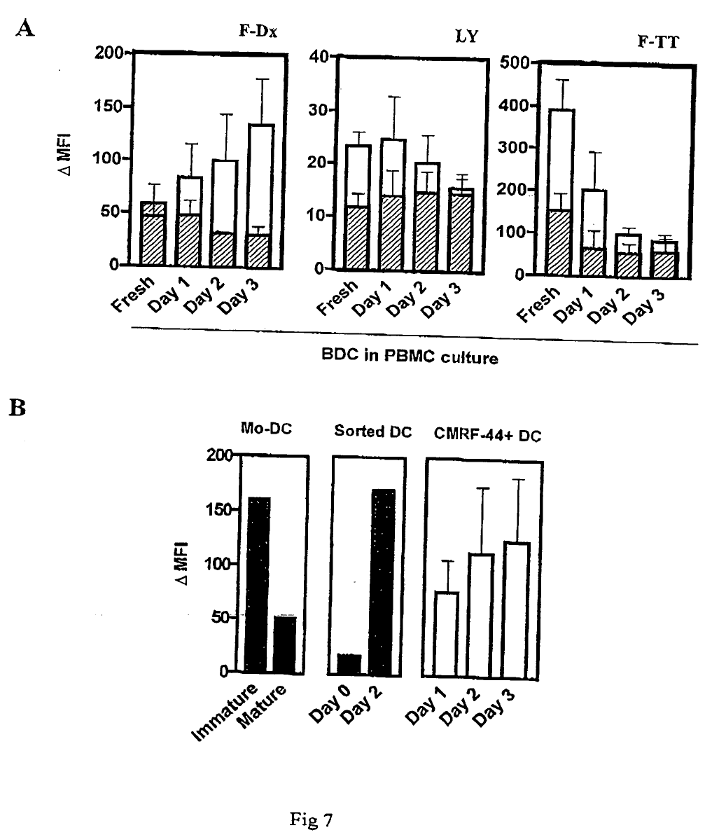

Next, we tested the antigen uptake capacity of the DCs within PBMC. Previous reports have shown that TNF-α differentiated/activated Mo-DCs and cultured Langerhans cells down regulate their antigen uptake capacity and their increase allo-stimmulatory activity (Sallusto F, Cella M, Danieli C, Lanzavecchia A. J Exp Med. 1995;182:389-400). In direct contrast to this, the culture and activation of DCs in PBMC preparations increased their uptake capacity of FITC-labeled dextran (F-Dx) (FIG. 7A, left). The uptake of the soluble agent Lucifer yellow (LY) did not change greatly during culture (FIG. 7A, middle). The greatest uptake of FITC-labeled tetanus toxoid (F-TT) occurred with fresh DCs, and thereafter decreased progressively with culture and activation (FIG. 7A, right). In each test system, the HLA-DR^{hi} population (FIG. 7A, open bars) appeared to have better antigen uptake capacity than the HLA-DR^{lo} population (FIG. 7A, hatched bars).

Mo-DCs were then generated and confined various reports (Sallusto F, Cella M, Danieli C, Lanzavecchia A. J Exp Med. 1995;182:389-400; Vuckovic S, Fearnley D B, Mannering S I, et al. Exp Hematol. 1998;26:1255-1264; Kato M, Neil T K, Fearnley D B, et al. Int Immunol. 2000;12: 151-1519) that their F-Dx uptake capacity was dramatically reduced when they become differentiated/activated (FIG. 7B, left). However, the cultured sorted Lin⁻ HLA-DR⁺ blood DCs increased their uptake levels to that similar to undifferentiated Mo-DCs. Freshly sorted Lin⁻ HLA-DR⁺ DCs took up F-Dx poorly, but after activation in culture, dramatically increased F-Dx uptake capacity (FIG. 7B, middle). Both DC subsets increased F-Dx uptake upon culture, with the CD11c⁺ population taking up at least 25 times more material (data not shown). DCs defined by the CMRF-44 antigen expression after overnight culture, also improved their F-Dx uptake with extended in vitro culture (FIG. 7B, right).

### EXAMPLE 8

### DCS Isolated from Cultured PBMC are Efficient Stimulators in the MLR

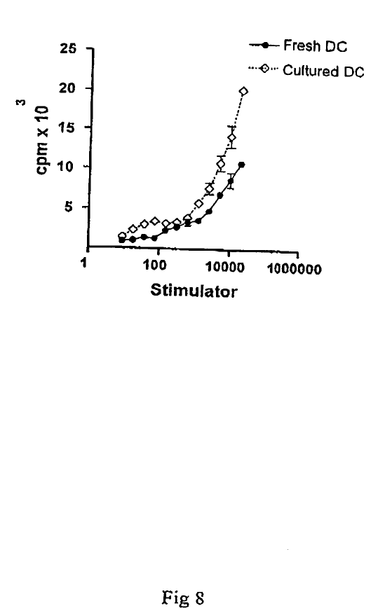

Finally, to test their co-stimulatory function, DCs were sorted from PBMC after 3 days of in vitro culture, and tested for their allo-stimulatory capacity. DCs cultured with PBMC were as potent stimulators in the allogeneic MLR as freshly isolated DCs (FIG. 8).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in tills specification, individually or collectively, and any and all combinations of any taco or more of said steps or features.

Bibliography

Cella M, Facchetti F, Lanzavecchia A, Colonna M. Nat Immunol. 2000;1:305-310.
Dranoff G, Crawford A D, Sadelain M, et al. Science. 1994;264:713-716.
Dzionek A, Fuchs A, Schmidt P, et al. J Immunol. 2000;165:6037-6046.
Fearnley D B, McLellan A D, Mannering S I, Hock B D, Hart D N J. Blood. 1997;89:3708-3716.
Fong L, Engleman E G. Annu Rev Immunol. 2000;18.245-273.
Hart D N J. Blood. 1997;90:3245-3287.
Kato M, Neil T K, Fearnley D B, et al. Int Immunol. 2000;12:1511-1519.
Kohrgruber N, Halanek N, Groger M, et al. J Immunol. 1999;163:3250-3259.
MacDonald Y P A, Munster D, Clark G, Vuckovic S, Hart D N J: Peripheral blood dendritic cell subset analysis, in Mason D (ed): Leucocyte Typing VII, vol. 7. Oxford, Oxford University Press, 2001
Mannering S I, McKenzie J L, Hart D N. J Immunol Methods. 1998;219:69-83.
Markowicz S, Engleman E G. J Clin Invest. 1990;85:955-961.
Nicholson J K, Stein D, Mui T, et al. Clin Diagn Lab Immunol. 1997;4:309-313.
Randolph G J, Inaba K, Robbiani D F, Steinman R M, Muller W A. Immunity. 1999;11:753-761.
Robinson S P, Patterson S, English N, et al. Eur J Immunol. 1999;29:2769-2778.
Sallusto F, Cella M, Danieli C, Lanzavecchia A. J Exp Med. 1995;182:389-400.
Siegal F P, Kadowaki N, Shodell M, et al. Science. 1999;284:1835-1837.
Vuckovic S, Fearnley D B, Mannering S I, et al. Exp Hematol. 1998;26:1255-1264.

1. A method of supporting the viability, proliferation or differentiation of mammalian dendritic cells, comprising culturing dendritic cells or precursors thereof in the presence of an effective number of mononuclear immune cells for a time and under conditions sufficient for said mononuclear immune cells to support said dendritic cells or precursors thereof.

2. The method according to claim 1 wherein said mononuclear immune cells are peripheral blood mononuclear cells.

3. The method according to claim 2 wherein said peripheral blood mononuclear cells are a whole peripheral blood mononuclear cell population.

4. The method according to any one of claims 1-3 wherein said dendritic cells or precursors thereof are blood-derived dendritic cells or precursors thereof.

5. The method according to claim 4 wherein said blood-derived dendritic cell precursors are CD14⁺ or CD16⁺ blood-derived dendritic cell precursors.

6. The method according to claim 5 wherein said method supports the differentiation of said CD 14⁺ or CD16⁺ blood-derived dendritic cell precursors to Lin⁻/HLA-DR⁺ dendritic cells.

7. The method according to claim 6 wherein said Lin⁻/HLA-DR⁺ dendritic cells are CD11c⁻/CD123^{hi} dendritic cells.

8. The method according to claim 6 wherein said Lin⁻/HLA-DR⁺ dendritic cells are CD11c⁺/CD123^{lo} dendritic cells.

9. The method according to claim 4 wherein said method supports the differentiation of said dendritic cells or precursors thereof and wherein expression of CD40, CD80, CD86, CMRF-44, CMRF-56 or CD83 is up-regulated.

10. The method according to claim 9 wherein said differentiated dendritic cells are CD11c⁻/CD123^{hi}.

11. The method according to claim 9 wherein said differentiated dendritic cells are CD11c⁺/CD123^{lo}.

12. The method according to claim 4 wherein said method supports the proliferation of said dendritic cells or precursors thereof.

13. The method according to claim 12 wherein said differentiated dendritic cells are CD11c⁻/CD123^{hi}.

14. The method according to claim 12 wherein said differentiated dendritic cells are CD11c⁺/CD123^{lo}.

15. A composition comprising tissue culture supernatant harvested from mononuclear immune cells cultured in accordance with the method of claim 4, wherein said culture supernatant supports the viability, proliferation or differentiation of mammalian dendritic cells or precursors thereof.

16. A method of producing dendritic cells, said method comprising culturing dendritic cells or precursors thereof in the presence of an effective number of mononuclear immune cells for a time and under conditions sufficient to produce dendritic cells or precursors thereof.

17. The method according to claim 16 wherein said mononuclear immune cells are peripheral blood mononuclear cells.

18. The method according to claim 17 wherein said peripheral blood mononuclear cells are a whole peripheral blood mononuclear cell population.

19. The method according to any one of claims 16-18 wherein said dendritic cells or precursors thereof are blood-derived dendritic cells or precursors thereof.

20. The method according to claim 19 wherein said blood-derived dendritic cell precursors are CD14⁺ or CD16⁺ blood-derived dendritic cell precursors.

21. The method according to claim 20 wherein said method supports the differentiation of said CD 14⁺ or CD16⁺ blood-derived dendritic cell precursors to Lin⁻/HLA-DR⁺ dendritic cells.

22. The method according to claim 21 wherein said Lin⁻/HLA-DR⁺ dendritic cells are CD11c⁻/CD123^{hi} dendritic cells.

23. The method according to claim 21 wherein said Lin⁻/HLA-DR⁺ dendritic cells are CD11c⁺/CD123^{lo} dendritic cells.

24. The method according to claim 19 wherein said method supports the differentiation of said dendritic cells or precursors thereof and wherein expression of CD40, CD80, CD86, CMRF-44, CMRF-56 or CD83 is up-regulated.

25. The method according to claim 24 wherein said differentiated dendritic cells are CD11c⁻/CD123^{hi}.

26. The method according to claim 24 wherein said differentiated dendritic cells are CD11c⁺/CD123^{lo}.

27. The method according to claim 19 wherein said method supports the proliferation of said dendritic cells or precursors thereof.

28. The method according to claim 27 wherein said differentiated dendritic cells are CD11c⁻/CD123^{hi}.

29. The method according to claim 27 wherein said differentiated dendritic cells are CD11c⁺/CD123^{lo}.

30-44. (canceled)
